(19) United States
(12) Patent Application Publication
(10) Pub. No.: US 2025/0367428 A1
(43) Pub. Date: Dec. 4, 2025

(54) DRIVING MECHANISM AND BLOOD PUMP

(71) Applicant: SHENZHEN CORE MEDICAL TECHNOLOGY CO., LTD, Shenzhen, Guangdong (CN)

(72) Inventors: Shunzhou YU, Shenzhen (CN); Duanqing XIE, Shenzhen (CN)

(21) Appl. No.: 19/108,104

(22) PCT Filed: Oct. 7, 2023
(86) PCT No.: PCT/CN2023/123248

§ 371 Date: Feb. 28, 2025

(30) Foreign Application Priority Data
Sep. 2, 2022 (CN) 202211072218.9

(51) Int. Cl. A61M 60/492; A61M 60/135; A61M 60/855; A61M 60/90

(52) U.S. Cl. CPC A61M 60/492; A61M 60/135; A61M 60/855; A61M 60/90; A61M 2205/103; A61M 2206/20

(57) ABSTRACT

A driving mechanism and a blood pump are disclosed. The driving mechanism comprises a housing assembly, a rotating assembly, and a sphere. The rotating assembly has a distal end and a proximal end; the distal end of the rotating assembly is rotatably mounted to the housing assembly. A first groove is formed on the proximal end of the rotating assembly, and the first groove has an internally concave first spherical wall. A second groove is formed on the housing assembly, and the second groove is arranged opposite the first groove; the second groove has an internally concave second spherical wall. A portion of the sphere is arranged within the first groove and a portion within the second groove, which are capable of sliding engagement with the first spherical wall and the second spherical wall, respectively.

This application claims priority to Chinese Patent Application No. 202211072218.9 filed with China National Intellectual Property Administration on Sep. 2, 2022, the entire disclosure of which is incorporated herein by reference.

### TECHNICAL FIELD

The present disclosure relates to the technical field of medical instruments, and in particular, to a driving mechanism and a blood pump.

### BACKGROUND

A blood pump is designed to be percutaneously inserted into a blood vessel of a patient, such as a blood vessel of an artery or vein of the thigh or armpit, and may be advanced into the heart of the patient to function as a left ventricular assist device or a right ventricular assist device. Therefore, the blood pump may also be referred to as an intracardiac or intravascular blood pump.

Generally, the blood pump has a driving mechanism and an impeller. The impeller is connected to a rotating assembly of the driving mechanism. In order to realize stable rotation of the rotating assembly, a structure for positioning or limiting the rotating assembly is usually required to be provided, so that the driving mechanism is complex in structure.

### SUMMARY

Accordingly, the present disclosure provides a driving mechanism and a blood pump having simple structures.

In a first aspect, embodiments of the present disclosure provide a driving mechanism, the driving mechanism including:
a housing assembly;
a rotating assembly having a distal end and a proximal end, the distal end of the rotating assembly being rotatably mounted on the housing assembly, the proximal end of the rotating assembly being provided with a first groove, and the first groove having a concave first spherical wall, wherein the housing assembly is provided with a second groove, the second groove being opposed to the first groove, and the second groove having a concave second spherical wall; and
a sphere partially arranged in the first groove and partially arranged in the second groove, and the sphere respectively slidably abutting against the first spherical wall and the second spherical wall.

In a second aspect, embodiments of the present disclosure provide a blood pump, including an impeller and a driving mechanism, the driving mechanism including:
a housing assembly;
a rotating assembly having a distal end and a proximal end, the distal end of the rotating assembly being rotatably mounted on the housing assembly, the proximal end of the rotating assembly being provided with a first groove, and the first groove having a concave first spherical wall, wherein the housing assembly is provided with a second groove, the second groove being opposed to the first groove, and the second groove having a concave second spherical wall; and
a sphere partially arranged in the first groove and partially arranged in the second groove, and wherein the sphere respectively slidably abuts against the first spherical wall and the second spherical wall; the impeller is connected to the rotating assembly, and rotatable along with the rotating assembly.

Details of one or more embodiments of the present disclosure are set forth in the accompanying drawings and the description below. Other features, objects, and advantages of the present disclosure will be apparent from the description, the accompanying drawings, and the claims.

### BRIEF DESCRIPTION OF THE DRAWINGS

In order to illustrate the technical solutions in the embodiments of the present disclosure more clearly, the drawings required for describing the embodiments or the prior art will be described briefly below. Apparently, the following described drawings are merely for some embodiments of the present disclosure, and other drawings can be derived from these drawings by those of ordinary skill in the art without any creative effort.

FIG. 1 is a schematic structural view of a blood pump according to an embodiment of the present disclosure;

FIG. 2 is a sectional view of the blood pump shown in FIG. 1 with an impeller, a stator, a cannula, and a part of a catheter omitted;

FIG. 3 is a sectional view of the blood pump in FIG. 1 in which a rotating shaft, a stop member, a rotor, a shaft sleeve, a sphere, and a support member are assembled together;

FIG. 4 is a partial enlarged view of portion I shown in FIG. 2;

FIG. 5 is a schematic structural view of the blood pump shown in FIG. 1 in which the rotor, the stator, and a magnetic conductive member are assembled together;

FIG. 6 is a schematic structural view of a first flywheel of a first rotor unit of the rotor shown in FIG. 2;

FIG. 7 is a schematic structural view showing a second stator unit shown in FIG. 6 and a magnetic conductive plate of the magnetic conductive member being assembled together;

FIG. 8 is a schematic structural view of the support member of the blood pump shown in FIG. 2;

FIG. 9 is a schematic sectional structural view of the support member shown in FIG. 8;

FIG. 10 is a schematic structural view of the first flywheel in FIG. 2;

FIG. 11 is a schematic structural view of the stop member of the blood pump shown in FIG. 2;

FIG. 12 is a sectional view of the blood pump shown in FIG. 1 from another perspective, with a part of the catheter omitted;

FIG. 13 is a partial enlarged view of portion II of FIG. 12;

FIG. 14 is a schematic structural view of a support base of the blood pump shown in FIG. 12;

FIG. 15 is a partial enlarged view of the blood pump shown in FIG. 2; and

FIG. 16 is a partial enlarged view of the shaft sleeve of the blood pump shown in FIG. 2.

### DETAILED DESCRIPTION

The present disclosure will be described in further detail below with reference to the accompanying drawings and embodiments in order to make the objects, technical solutions, and advantages of the present disclosure more clear. It should be understood that the specific embodiments described herein are only for explaining the present disclosure, and not intended to limit the present disclosure.

It should be noted that when an element is referred to as being “fixed on” or “provided at” another element, the element may be directly or indirectly located on the other element. When an element is referred to as being “connected to” another element, the element may be directly or indirectly connected to the other element.

In addition, the terms such as “first” and “second” are used herein for purposes of description and are not intended to indicate or imply relative importance or significance or to imply the number of indicated technical features. Thus, the feature defined by “first” and “second” may include one or more of the features explicitly or implicitly. In the description of the present disclosure, “a plurality of” means two or more, unless otherwise explicitly specified.

In order to illustrate the technical solution of the present disclosure, the following description is given with reference to the specific drawings and embodiments.

In the field of interventional medical treatment, it is common to define the end of an instrument proximate to an operator as the proximal end and the end away from the operator as the distal end.

A driving mechanism 10 and a blood pump 1 according to embodiments of the present disclosure are described now.

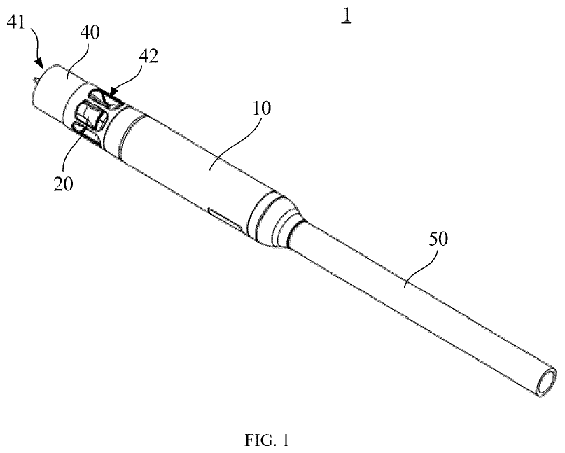

Referring to FIG. 1, the blood pump 1 includes a driving mechanism 10 and an impeller 20. The driving mechanism 10 is in transmission connection to the impeller 20, and the driving mechanism 10 can drive the impeller 20 to rotate.

Specifically, the blood pump 1 further includes a cannula 40. The cannula 40 is fixedly connected to a distal end of the driving mechanism 10. The impeller 20 is rotatably received in the cannula 40. The cannula 40 has a blood outlet 42 and a blood inlet 41. When the impeller 20 rotates, blood flows into the cannula 40 via the blood inlet 41 and then flows out via the blood outlet 42. In an embodiment, the cannula 40 extends through a heart valve 10 such as an aortic valve, the blood inlet 41 is located in the heart. The blood outlet 42 and the driving mechanism 10 are located in a blood vessel outside the heart, such as the aorta.

Specifically, the blood pump 1 further includes a catheter 50, the catheter 50 being connected to a proximal end of the driving mechanism 10. The catheter 50 is configured to receive various supply lines. For example, the supply lines include a wire for being electrically connected to the driving mechanism 10 and a flushing line for passing flushing liquid to the blood pump 1. Optionally, the flushing liquid is physiological saline, physiological saline containing heparin, glucose, or the like.

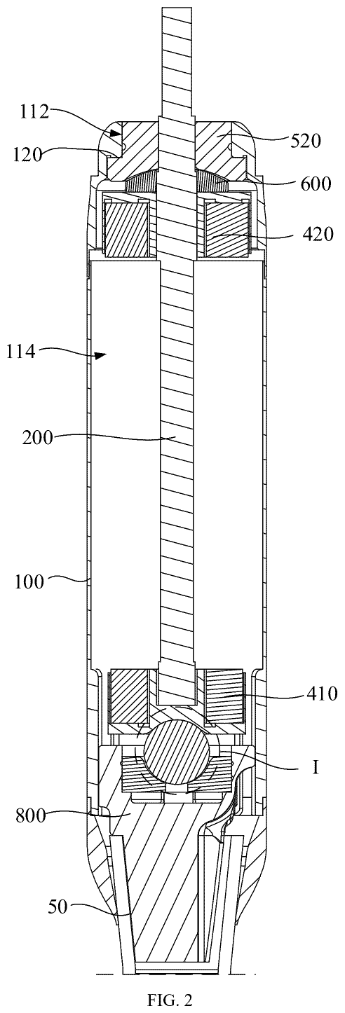

Referring to FIG. 2 to FIG. 7, the driving mechanism 10 includes a pump case 100, a rotating shaft 200, a rotor 400, a support member 510, a shaft sleeve 520, and a sphere 900. The pump case 100, the shaft sleeve 520, and the support member 510 form a housing assembly; the rotating shaft 200 and the rotor 400 form a rotating assembly; the rotating assembly is rotatably mounted on the housing assembly, and configured to be connected to the impeller 20 to drive the impeller 20 to rotate. The rotating assembly has a distal end and a proximal end. The distal end of the rotating assembly is rotatably mounted to the housing assembly. The proximal end of the rotating assembly is provided with a first groove 4124. The first groove 4124 has a concave first spherical wall 4124a. The housing assembly is provided with a second groove 512. The second groove 512 has a concave second spherical wall 514. The second groove 512 is opposed to the first groove 4124. The sphere 900 is partially arranged in the first groove 4124 and slidably abuts against the first spherical wall 4124a, and partially arranged in the second groove 512 and slidably abuts against the second spherical wall 514, so that the sphere 900 is limited and supported. The sphere 900, the first groove 4124, and the second groove 512 cooperate with one another to support and limit the proximal end of the rotating assembly. Meanwhile, since a radial rolling path of the sphere 900 is limited by the first groove 4124 and the second groove 512, a radial swinging range of the rotating assembly is limited. Finally, since the sphere 900, the rotating assembly, and the housing assembly are independent from one another, in an assembling process, only a rotating axis of the rotating assembly is required to coincide with a central axis of a cavity defined by the first spherical wall 4124a, a central axis of the sphere 900 can be ensured to coincide with the rotating axis of the rotating assembly by arranging the sphere 900 in the first groove 4124. The housing assembly then cooperates with the sphere 900. The second groove 512 is only required to support and limit the sphere 900, and a central axis of a cavity defined by the second spherical wall 514 is not required to coincide with the central axis of the sphere 900, thus reducing assembling difficulty.

The pump case 100 is substantially of a cylindrical structure with both ends provided with openings. A distal end of the pump case 100 is fixedly connected to the cannula 40, and a proximal end thereof is fixedly connected to the catheter 50. The pump case 100 has an inner cavity. Specifically, the inner cavity is divided into a limiting cavity 112 and a receiving cavity 114. In the illustrated embodiment, the limiting cavity 112 and the receiving cavity 114 are arranged in an axial direction of the pump case 100.

The rotating shaft 200 is rotatably mounted to the pump case 100, the rotating shaft 200 having a connecting end 210 for being connected to the impeller 20. In the illustrated embodiment, the rotating shaft 200 extends substantially in the axial direction of the pump case 100. Alternatively, an extending direction of an axis of the rotating shaft 200 is substantially coincident with the axial direction of the pump case 100. The limiting cavity 112 and the receiving cavity 114 are arranged along the axis of the rotating shaft 200. The rotating shaft 200 extends through the limiting cavity 112, partially received in the receiving cavity 114, and partially located outside the pump case 100 or partially extends into the cannula 10. The part of the rotating shaft 200 extending outside the pump case 100 or extending into the cannula 10 is the connecting end 210 of the rotating shaft 200. Specifically, the impeller 20 is fixedly connected to the connecting end 210, so that the impeller 20 can rotate along with the rotating shaft 200.

The rotor 400 is located in the pump case 100. That is, the rotor 400 is also arranged in the inner cavity of the pump case 100. In the illustrated embodiment, the rotor 400 is located within the receiving cavity 114. The rotor 400 is fixedly connected to the rotating shaft 200. The first groove 4124 is located on one of the rotating shaft 200 and the rotor 400.

The driving mechanism 10 further includes a stator 300. The stator 300 can drive the rotating assembly to rotate. Specifically, the stator 300 can drive the rotor 400 to rotate, and the rotor 400 can drive the rotating shaft 200 to rotate. More specifically, the rotor 400 has magnetic property, and the stator 300 is capable of generating a rotating magnetic field to drive the rotor 400 to rotate. The stator 300 is fixedly mounted to the pump case 100. That is, the stator 300 is arranged in the inner cavity of the pump case 100. In the illustrated embodiment, the stator 300 is located in the receiving cavity 114. The rotating shaft 200 rotatably extends through the stator 300.

Referring to FIG. 5 together, in the illustrated embodiment, the rotor 400 includes a first rotor unit 410 and a second rotor unit 420. The first rotor unit 410 and the second rotor unit 420 are both fixedly connected to the rotating shaft 200. Both the first rotor unit 410 and the second rotor unit 420 are rotatably received in the receiving cavity 114 of the pump case 100. The first rotor unit 420 and the second rotor unit 420 are arranged along the axis of the rotating shaft 200. The stator 300 is located between the first rotor unit 410 and the second rotor unit 420. Both the first rotor unit 410 and the second rotor unit 420 have magnetic property. The stator 300 is capable of generating a rotating magnetic field to drive the first rotor unit 410 and the second rotor unit 420 to rotate.

Specifically, the first rotor unit 410 includes a first magnet 411. The first magnet 411 is fixedly connected to the rotating shaft 200. The first magnet 411 is a ring-shaped Halbach array magnet.

Specifically, the first rotor unit 410 further includes a first flywheel 412. The first flywheel 412 is fixedly connected to the rotating shaft 200. The first magnet 411 is fixedly connected to the first flywheel 412. The arrangement of the first flywheel 412 can enhance connecting strength of the first magnet 411 and the rotating shaft 200, and can further reduce shaking of the rotating shaft 200 in a rotating process, so that the whole rotating shaft 200 is more stable in the rotating process. In the illustrated embodiment, the first groove 4124 is located on the first rotor unit 410, specifically, on the first flywheel 412.

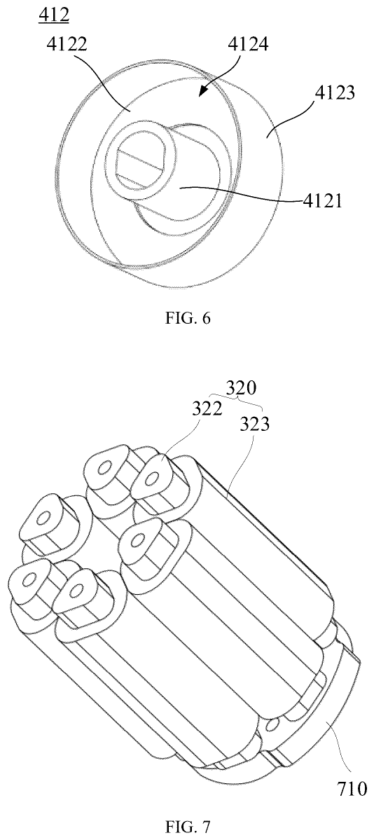

Referring to FIG. 6 together, specifically, the first flywheel 412 includes a first built-in tube 4121, a first disc portion 4122, and a first outer annular wall 4123. Both the first built-in tube 4121 and the first outer annular wall 4123 are of a circular tubular structure. The first disc portion 4122 is of an annular disc structure. Both the first built-in tube 4121 and the first outer annular wall 4123 are fixedly connected to the first disc portion 4122. The first outer annular wall 4123 is arranged surrounding the first disc portion 4122. The first built-in tube 4121 and the first outer annular wall 4123 are coaxially arranged. The rotating shaft 200 is sleeved with the first built-in tube 4121 and fixedly connected to the first built-in tube 4121. A first annular cavity 4124 is formed between the first built-in tube 4121 and the first outer annular wall 4123. The first magnet 411 is received in the first annular cavity 4124. The first annular cavity 4124 is shaped to be adapted to the first magnet 411 to facilitate mounting and positioning of the first magnet 411. Such an arrangement enables the first flywheel 412 to limit the first magnet 411, so that the first magnet 411 can be conveniently mounted, and the first magnet 411 and the first flywheel 412 can be combined more stably.

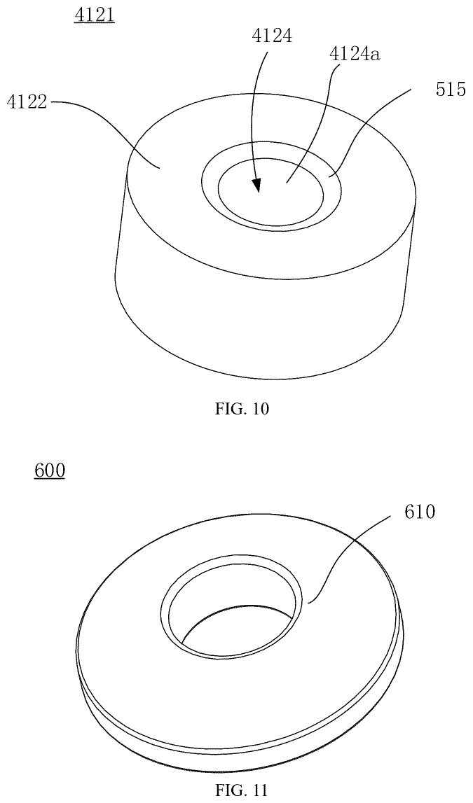

Referring to FIG. 10 together, the first groove 4124 is arranged at a center of the first disc portion 4122, and the first built-in tube 4121 is also arranged at the center of the first disc portion 4122, in other words, a central axis of the first groove 4124 coincides with a central axis of the first built-in tube 4121. In this embodiment, a proximal end of the rotating shaft 200 is received in the first built-in tube 4121 and fixedly connected to the first built-in tube 4121, but does not extend out of the first disc portion 4122, so as to facilitate assembling and positioning of the rotating shaft 200 and the first rotor unit 410. A central axis of the rotating shaft 200 coincides with the central axis of the first built-in tube 4121.

It should be noted that the first flywheel 412 is not limited to have the above structure, and in some embodiments, the first flywheel 412 does not have the first outer annular wall 4123. In some embodiments, the first flywheel 412 does not have the first outer annular wall 4123 and the first built-in tube 4121, and in this case, the rotating shaft 200 fixedly extends through the center of the first disc portion 4122, and the first groove 4124 may be arranged at an end portion of the proximal end of the rotating shaft 200. The arrangement of the first built-in tube 4121 enables the first flywheel 412 to be more stably connected to the rotating shaft 200 compared with the first flywheel 412 having only the first disc portion 4122.

The second rotor unit 420 includes a second magnet 421. The second magnet 421 is fixedly connected to the rotating shaft 200. Specifically, the second magnet 421 is a ring-shaped Halbach array magnet.

Specifically, the second rotor unit 420 further includes a second flywheel 422. The second flywheel 422 is fixedly connected to the rotating shaft 200, and the second magnet 421 is fixed to the second flywheel 422. The arrangement of the second flywheel 422 can enhance connecting strength between the second magnet 421 and the rotating shaft 200, and can further reduce shaking of the rotating shaft 200 in the rotating process, so that the whole rotating shaft 200 is more stable in the rotating process.

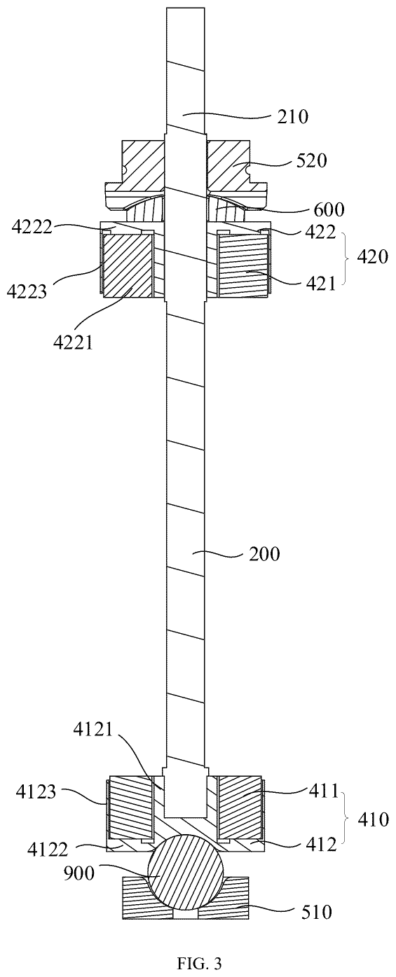

Specifically, referring to FIG. 3, the second flywheel 422 includes a second built-in tube 4221, a second disc portion 4222, and a second outer annular wall 4223. Both the second built-in tube 4221 and the second outer annular wall 4223 are of a circular tubular structure, and the second disc portion 4222 is of an annular disc structure. Both the second built-in tube 4221 and the second outer annular wall 4223 are fixedly connected to the second disc portion 4222. The second outer annular wall 4223 is arranged surrounding the second disc portion 4222. The second built-in tube 4221 and the second outer annular wall 4223 are coaxially arranged.

The rotating shaft 200 extends through the second built-in tube 4221 and fixedly connected to the second built-in tube 4221. A second annular cavity is formed between the second built-in tube 4221 and the second outer annular wall 4223. The second magnet 421 is received in the second annular cavity. The second annular cavity is shaped to be adapted to the second magnet 421 to facilitate mounting and positioning of the second magnet 421. Such an arrangement enables the second flywheel 422 to limit the second magnet 421, so that the second magnet 421 can be conveniently mounted, and the second magnet 421 and the second flywheel 422 can be combined more stably.

It should be noted that the second flywheel 422 is not limited to have the above structure, and in some embodiments, the second flywheel 422 does not have the second outer annular wall 4223. In some embodiments, the second flywheel 422 does not have the second outer annular wall 4223 and the second built-in tube 4221, and in this case, the rotating shaft 200 fixedly extends through a center of the second disc portion 4222. The arrangement of the second built-in tube 4221 enables the second flywheel 422 to be more stably connected to the rotating shaft 200 compared with the second flywheel 422 having only the second disc portion 4222.

Specifically, the stator 300 includes a first stator unit 310 and a second stator unit 320 that are arranged along the axis of the rotating shaft 200. The first stator unit 310 can drive the first rotor unit 410 to rotate, and the second stator unit 320 can drive the second rotor unit 420 to rotate. Specifically, the first stator unit 310 can generate a rotating magnetic field to drive the first rotor unit 410 to rotate, and the second stator unit 320 can generate a rotating magnetic field to drive the second rotor unit 420 to rotate. Both the first stator unit 310 and the second stator unit 320 are fixedly received in the receiving cavity 114 of the pump case 100. The rotating shaft 200 rotatably extends through the first stator unit 310 and the second stator unit 320. Both the first stator unit 310 and the second stator unit 320 are located between the first rotor unit 410 and the second rotor unit 420.

Each of the first stator unit 310 and the second stator unit 320 includes a magnetic core and a coil wound around the magnetic core. Specifically, the first stator unit 310 includes a first magnetic core 312 and a first coil 313. The first coil 313 is wound around the first magnetic core 312. A plurality of first magnetic cores 312 are provided. The plurality of first magnetic cores 312 are arranged in a circle around the axis of the rotating shaft 200. Each first magnetic core 312 is provided with one first coil 313.

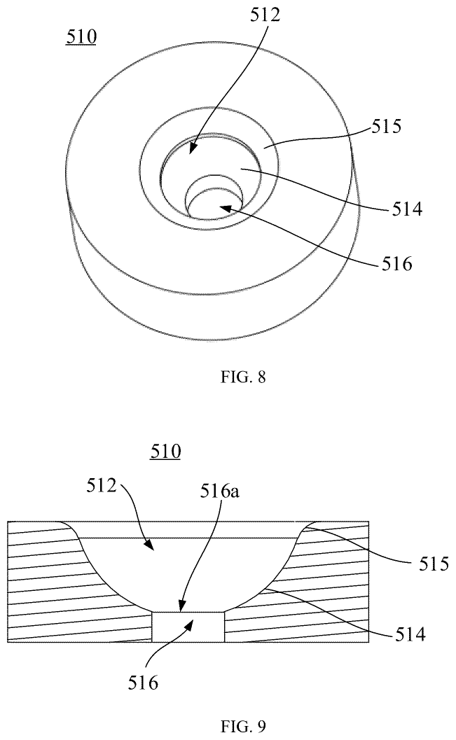

The second stator unit 320 has a structure similar to that of the first stator unit 310. Referring to FIG. 8 together, the second stator unit 320 includes a second magnetic core 322 and a second coil 323. The second coil 323 is wound around the second magnetic core 322. A plurality of second magnetic cores 322 are provided. The plurality of second magnetic cores 322 are arranged in a circle around the axis of the rotating shaft 200. Each second magnetic core 322 is provided with one second coil 323.

Specifically, the driving mechanism 10 further includes a magnetic conductive member 700 connected to the pump case 100. Both the first magnetic core 312 of the first stator unit 310 and the second magnetic core 322 of the second stator unit 320 are fixedly connected to the magnetic conductive member 700. Specifically, the magnetic conductive member 700 is fixedly received in the pump case 100, for example, engaged, welded or bonded to an inner sidewall of the pump case 100. The rotating shaft 200 rotatably extends through the magnetic conductive member 700. An end of the first magnetic core 312 is fixedly connected to the magnetic conductive member 700, and the first rotor unit 410 is arranged proximate to another end of the first magnetic core 312. An end of the second magnetic core 322 is fixedly connected to the magnetic conductive member 700, and the second rotor unit 420 is arranged proximate to another end of the second magnetic core 322.

The magnetic conductive member 700 functions to close a magnetic circuit, so as to promote and increase generation of a magnetic flux, improving a coupling capability, and therefore, the provided magnetic conductive member 700 can function to close a magnetic circuit between the first stator unit 310 and the first rotor unit 410, close a magnetic circuit between the second stator unit 320 and the second rotor unit 420, increasing the magnetic flux, and thus, the arrangement of the magnetic conductive member 700 is beneficial to reducing an overall diameter of the driving mechanism 10. In addition, both the first magnetic core 312 of the first stator unit 310 and the second magnetic core 322 of the second stator unit 320 are fixedly connected to the magnetic conductive member 700, so that positioning and mounting of the first stator unit 310 and the second stator unit 320 can be realized, thus reducing the assembling difficulty of the first stator unit 310 and the second stator unit 320. Meanwhile, the magnetic conductive member 700 arranged in the above manner can also avoid the arrangement of a positioning structure in the pump case 100, thereby simplifying the structure of the pump case 100 and simplifying the assembling process of the whole driving mechanism 10.

Specifically, the magnetic conductive member 700 includes two magnetic conductive plates 710. The two magnetic conductive plates 710 are stacked together. One of the magnetic conductive plates 710 is fixedly connected to the first magnetic core 312 of the first stator unit 310, another of the magnetic conductive plates 710 is fixedly connected to the second magnetic core 322 of the second stator unit 320, and the rotating shaft 200 rotatably extends through the two magnetic conductive plates 710. Specifically, the two magnetic conductive plates 710 are separate before being assembled. By configuring the magnetic conductive member 700 into the two magnetic conductive plates 710 which are separate before being assembled, when the driving mechanism 10 is assembled, firstly, the first magnetic core 312 can be fixedly connected to the magnetic conductive plate 710, the second magnetic core 322 is fixedly connected to another magnetic conductive plate 710, and the two magnetic conductive plates 710 are then stacked together. In this way, the first magnetic core 312 and the second magnetic core 322 can be conveniently assembled to the two magnetic conductive plates 710 respectively, and the first magnetic core 321 and the second magnetic core 322 can be assembled more conveniently.

Specifically, the two magnetic conductive plates 710 are fixedly connected such that the first stator unit 310, the second stator unit 320, and the magnetic conductive member 700 forms a whole to be assembled into the pump case 100, which make the stator 300 be assembled more easily. For example, the two magnetic conductive plates 710 may be connected together by gluing or welding. It can be understood that in other embodiments, the two magnetic conductive plates 710 are not fixedly connected, but in contact with each other.

It should be noted that the magnetic conductive member 700 is not limited to be formed by combining the two separate magnetic conductive plates 710 in the above-mentioned manner. The magnetic conductive member 700 may be of a plate-shaped structure, and both the first magnetic core 312 and the second magnetic core 322 are connected to the magnetic conductive member 700. That is, the first stator unit 310 and the second stator unit 320 share one magnetic conductive member 700.

Specifically, the magnetic conductive plate 710 is made of silicon steel, and the first magnetic core 312 and the second magnetic core 322 are made of silicon steel.

The sphere 900 is movably received in the pump case 100. Specifically, the sphere 900 is located in the receiving cavity 114.

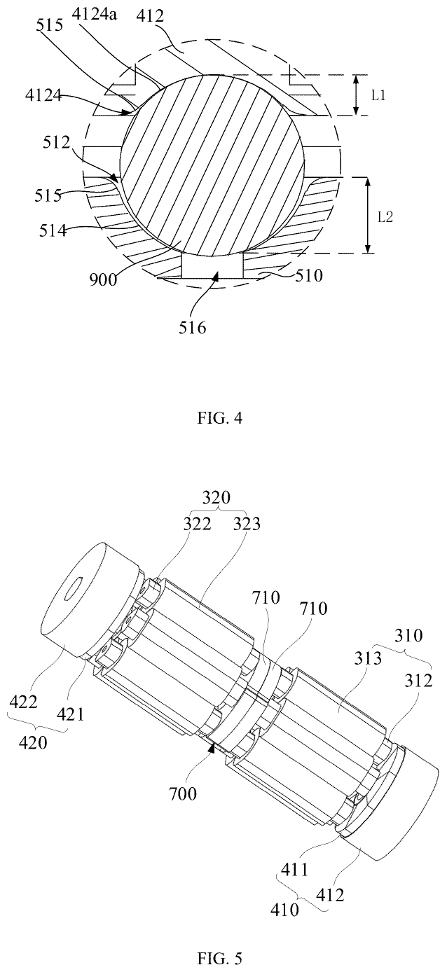

Referring again to FIG. 2, FIG. 3, and FIG. 4, the support member 510 and the shaft sleeve 520 are mounted within the pump case 100. Specifically, the support member 510 is received in the receiving cavity 114, and the shaft sleeve 520 is received in the limiting cavity 112. The support member 510 and the shaft sleeve 520 are fixedly connected to the pump case 100. The support member 510, the shaft sleeve 520, and the sphere 900 are arranged along the axial direction of the pump case 100. The support member 510, the shaft sleeve 520, and the sphere 900 can limit the rotating assembly together. The shaft sleeve 520 is closer to the connecting end 210 of the rotating shaft 200 than the support member 510. The rotor 400 is located between the sphere 900 and the shaft sleeve 520. The stator 300 is also located between the sphere 900 and the shaft sleeve 520. In the illustrated embodiment, the first rotor unit 410, the second rotor unit 420, the first stator unit 310, and the second stator unit 320 are all located between the sphere 900 and the shaft sleeve 520. The sphere 900 is located between the first rotor unit 410 and the support member 510. The first rotor unit 410 is arranged proximate to the sphere 900, and the second rotor unit 420 is arranged proximate to the shaft sleeve 520. In other words, the support member 510, the sphere 900, the first rotor unit 410, the first stator unit 310, the second stator unit 320, the second rotor unit 420, and the shaft sleeve 520 are sequentially arranged along the axis of the rotating shaft 200. The shaft sleeve 520 is closest to the connection end 210 of the rotating shaft 200. The second groove 512 is formed in the support member 510.

Referring to FIG. 8, FIG. 9, and FIG. 10 together, specifically, both an opening edge of the first groove 4124 proximate to the second groove 512 and an opening edge of the second groove 512 proximate to the first groove 4124 are provided with rounded portions 515 respectively. In the rotating process of the rotating shaft 200, the first rotor unit 410 away from the connecting end 210 may have a small radial deflection, which may drive the sphere 900 to roll radially in the first groove 4124 and the second groove 512. That is, the rounded portion 515 are arranged to prevent the sphere 900 from being scratched and abraded by the opening edge of the first groove 4124 having edges and corners and the opening edge of the second groove 512 having edges and corners.

Specifically, a diameter of the sphere 900 is greater than a sum of lengths of the first groove 4124 and the second groove 512 along the rotating axis of the rotating assembly (when the rotating assembly does not swing radially), so that the proximal end of the rotating assembly is spaced apart from the housing assembly (e.g., the support member 510) by a distance to avoid contact between the proximal end of the rotating assembly and the housing assembly when the rotating assembly swings radially. As shown in FIG. 4, L1 refers to the length of the first groove 4124 along the rotating axis of the rotating assembly, L2 refers to the length of the second groove 512 along the rotating axis of the rotating assembly (when the rotating assembly does not swing radially). Specifically, L2 refers to the length of the second groove 512 in the axial direction of the pump case 100. The sphere 900 is partially located outside the first groove 4124 and the second groove 512. An opening of the first groove 4124 at a side proximate to the second groove 512 and an opening of the second groove 512 at a side proximate to the first groove 4124 are spaced apart from each other by a distance. That is, the rotor 400 (specifically, the first rotor unit 410) and the support member 510 are spaced apart from each other by a distance, so as to prevent direct friction and abrasion between the rotor 400 and the support member 510.

More specifically, the length L1 of the first groove 4124 along the rotating axis of the rotating assembly is greater than or equal to ¼ of the diameter of the sphere 900 and less than ½ of the diameter of the sphere 900. In this way, a contact area between the sphere 900 and the first groove 4124 is within this range, thus ensuring that abrasion between the sphere 900 and the first spherical wall 4124a is within a reasonable range. If the length L1 is less than ¼ of the diameter of the sphere 900, the contact area between the sphere 900 and the first spherical wall 4124a is too small, resulting in too much abrasion. If the length L1 is greater than ½ of the diameter of the sphere 900, and meanwhile, the sphere 900 extends into the first groove 4124 too deeper, and is thus too firmly limited radially, the first spherical wall 4124a has a too steep slope, and radial rolling of the sphere 900 is difficult, resulting in that the sphere 900 has a reduced deflection adapting capability, and the rotating assembly does not rotate smoothly or even is jammed. Meanwhile, in the illustrated embodiment, since the first groove 4124 is formed in a side surface of the first disc portion 4122 away from the first magnet 411, if having too large depth, the first groove 4124 would interfere with a mounting space of the first magnet 411, it is necessary to increase a thickness of the first disc portion 4122 to resolve it, which increases an overall axial length of the rotating assembly, resulting in structural crowding in the entire pump case 100.

Similarly, the length L2 of the second groove 512 along the rotating axis of the rotating assembly (when the rotating assembly does not swing radially) is greater than or equal to ¼ of the diameter of the sphere 900 and less than ½ of the diameter of the sphere 900. By setting a contact area between the sphere 900 and the second groove 512 within this range, abrasion between the sphere 900 and the second spherical wall 514 is small. If the length L2 is less than ¼ of the diameter of the sphere 900, the contact area between the sphere 900 and the second spherical wall 514 is too small, resulting in too much abrasion. If the length L2 is greater than ½ of the diameter of the sphere 900, the sphere 900 extends into the second groove 512 too deeper, and is thus too firmly limited radially, the second spherical wall 514 has a too steep slope, and radial rolling of the sphere 900 is difficult, resulting in that the sphere 900 has a reduced deflection adapting capability, and the rotating assembly does not rotate smoothly or even is jammed,

Specifically, a diameter of a sphere where the first spherical wall 4124a is located is greater than the diameter of the sphere 900. Since the rotating assembly may generate the small radial deflection in the rotating process, the sphere 900 may be driven to roll along the first spherical wall 4124a. The diameter of the sphere where the first spherical wall 4124a is located is greater than the diameter of the sphere 900, that is, a distance between the first spherical wall 4124a and an outer wall of the sphere 900 gradually increases in a radial direction, so that the sphere 900 may not be completely covered in the radial direction. That is, the sphere 900 has a rollable space in the first groove 4124 to adapt to the deflection of the rotating shaft 200 to avoid from being jammed. Similarly, a diameter of a sphere where the second spherical wall 514 is located is greater than the diameter of the sphere 900. The diameter of the sphere where the second spherical wall 514 is located is greater than the diameter of the sphere 900, that is, a distance between the second spherical wall 514 and the outer wall of the sphere 900 gradually increases in the radial direction, so that the sphere 900 is not completely covered in the radial direction, and the sphere 900 has a rollable space in the second groove 512 to adapt to the deflection of the rotating shaft 200 to avoid from being jammed.

Specifically, the second groove 512 has a first opening 512a and a second opening 516a. The support member 510 is further provided with a communication hole 516 in communication with the second groove 512. The communication hole 516 is in communication with the second opening 516a. The communication hole 516 can be in communication with the flushing line in the catheter 50, so that the flushing liquid can enter the second groove 512 via the communication hole 516, and then flow into the receiving cavity 114 via the second groove 512. The flushing liquid enters and to be between the second spherical wall 514 of the second groove 512 and the sphere 900, which can achieve the functions of lubrication and heat dissipation, so as to reduce friction between the sphere 900 and the second spherical wall 514 of the second groove 512 and dissipate the generated heat, thus reducing abrasion between the sphere 900 and the second spherical wall 514.

Specifically, the first opening 512a is closer to the first groove 4124 than the second opening 516a, and the second opening 516a is centrally located at the second spherical wall 514, so that the flushing liquid entering the second groove 512 via the communication hole 516 provides an axial flushing force for the sphere 900 as much as possible. More specifically, a central axis of the communication hole 516 coincides with the central axis of the cavity defined by the second spherical wall 514. That is, a central axis of the first opening 512a coincides with a central axis of the second opening 516a. The communication hole 516 is a straight hole to reduce energy consumption of the flushing liquid in the communication hole 516.

Specifically, a diameter of the second opening 516a is 1/9 to ⅓ of the diameter of the sphere 900. In the illustrated embodiment, a hole size of the communication hole 516 is constant. That is, the hole size of the communication hole 516 is 1/9 to ⅓ of the diameter of the sphere 900. If the diameter of the opening 516a of the communication hole 516 in the second spherical wall 514 is too large, a contact surface between the sphere 900 and the second spherical wall 514 may be reduced, thus increasing the abrasion of the sphere 900 by the second spherical wall 514. If the diameter of the opening 516a is too small, an amount of the flushing liquid entering the second groove 512 via the communication hole 516 may be affected, and the flushing liquid entering the second groove 512, on the one hand, is required to give a flushing force to the sphere 900, and on the other hand, enters to be between the sphere 900 and the second spherical wall 514 to achieve a lubricating effect, to reduce a friction coefficient between the sphere 900 and the second spherical wall 514, and therefore, the amount of the flushing liquid entering the second groove 512 should not be too small.

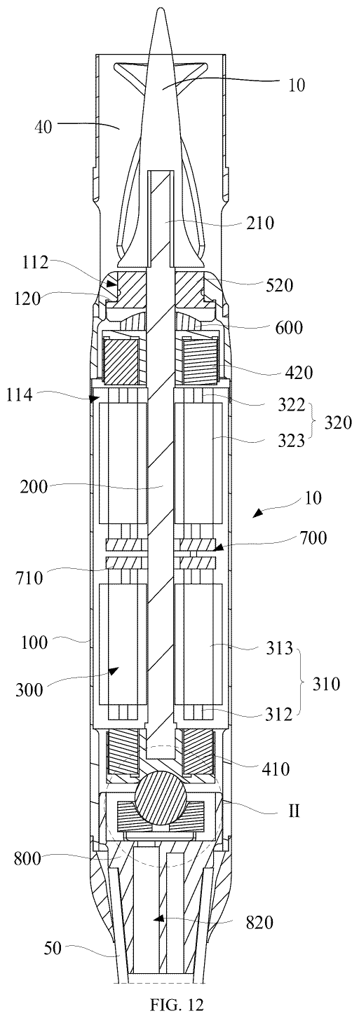

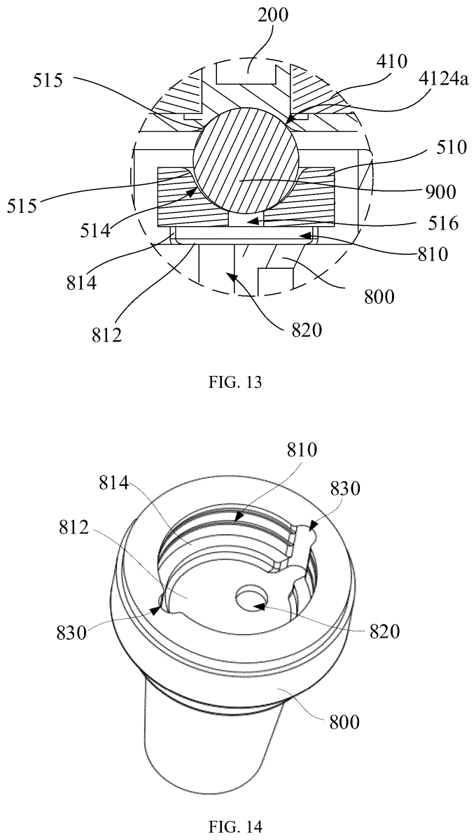

Referring to FIG. 12, FIG. 13, and FIG. 14 together, specifically, the driving mechanism 10 further includes a support base 800. The support base 800 is fixedly connected to the pump case 100. The support base 800 is provided with a mounting cavity 810 and a liquid passing hole 820 in communication with the mounting cavity 810. The support member 510 is mounted in the mounting cavity 810. The communication hole 516 has a certain length along the central axis of the first opening 512a. The communication hole 516 is communicated with the liquid passing hole 820. An end of the liquid passing hole 820 away from the mounting cavity 810 is configured to be communication with the flushing line of the catheter 50, so that the flushing liquid can flow through the liquid passing hole 820 and the communication hole 516, and then flow into a gap between the second spherical wall of the second groove 512 and the sphere 900, and then into the inner cavity of the pump case 100 via the first opening 512a.

After flowing out of the first opening 512a, the flushing liquid also flows into the first groove 4124. The flushing liquid enters to be between the first spherical wall 4124a of the first groove 4124 and the sphere 900, which can achieve the functions of lubrication and heat dissipation, to reduce friction between the sphere 900 and the first spherical wall 4124a of the first groove 4124 and dissipate the generated heat, thus reducing the abrasion between the sphere 900 and the first spherical wall 4124a.

Specifically, the mounting cavity 810 has a cavity bottom 812. An opening of the liquid passing hole 820 is located at the cavity bottom 812 of the mounting cavity 810. A support step 814 is arranged in the mounting cavity 810. The support step 814 abuts against the support member 510, so that the support member 510 is spaced apart from the cavity bottom 812 by a distance, so as to better guarantee smoothness of flowing of the flushing liquid. Specifically, the support step 814 abuts against a surface of the support member 510 facing away from the shaft sleeve 520.

Specifically, the support base 800 is further provided with a flow diversion channel 830. The flow diversion channel 830 is in fluid communication with the liquid passing hole 820, so that the flushing liquid flowing through the liquid passing hole 820 can also flow into the inner cavity of the pump case 100 through the flow diversion channel 830. Specifically, an end of the flow diversion channel 300 is in communication with a gap between the support member 510 and the cavity bottom 812 of the mounting cavity 810, and another end thereof is in communication with the receiving cavity 114. In the illustrated embodiment, the flow diversion channel 830 is formed by partially recessing a cavity wall of the mounting cavity 810. In other words, in a normal state, the flushing liquid is divided into two streams after entering the mounting cavity 810 via the liquid passing hole 820; one stream flows into the second groove 512 of the support member 510 via the communication hole 516, and another stream flows out via the flow diversion channel 830. The arrangement of the flow diversion channel 830 can ensure circulation of the flushing liquid in the case where the communication hole $16 is blocked by the sphere 900.

In the illustrated embodiment, two flow diversion channels 830 are provided. The two flow diversion channels 830 are arranged opposed to each other. It may be understood that the number of the flow diversion channels 830 can be adjusted according to design requirements. For example, in some embodiments, one or more than two flow diversion channels 830 can be provided.

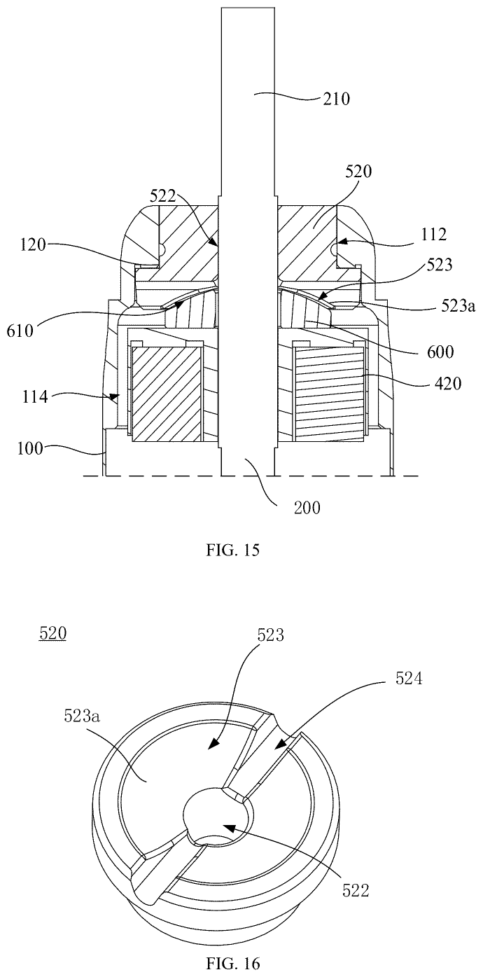

Referring to FIG. 2, FIG. 3, FIG. 11, FIG. 15, and FIG. 16 together, the shaft sleeve 520 is provided with a limiting step 120. In the illustrated embodiment, the limiting step 120 is formed by cutting a side surface of the shaft sleeve 520 proximate to the impeller 20 by a certain depth along the central axis of the rotating shaft 200. With the limiting step 120, the shaft sleeve 520 can be conveniently mounted on the pump case 100 and positioned, thus facilitating assembly of the shaft sleeve 520. The shaft sleeve 520 is provided with a shaft hole 522. The rotating shaft 200 rotatably extends through the shaft hole 522. In the illustrated embodiment, a central axis of the shaft hole 522 coincides with the central axis of the communication hole 516. A gap for fluid circulation is provided between a hole wall of the shaft hole 522 of the shaft sleeve 520 and the rotating shaft 200. The flushing liquid entering the receiving cavity 114 can flow through the gap between the rotating shaft 200 and the hole wall of the shaft hole 522 and flow out of the pump case 100.

A stop member 600 is fixedly connected to the rotating assembly. Specifically, the stop member 600 is fixedly connected to at least one of the rotating shaft 200 and the rotor 400 (specifically, the second rotor unit 420). In other words, the stop member 600 may be directly fixed only to the rotor 400, only to the rotating shaft 200, or to both the rotor 400 and the rotating shaft 200. Since the rotor 400 is fixedly connected to the rotating shaft 200, the stop member 600, the rotating shaft 200, and the rotor 400 rotate and move synchronously. The stop member 600 is located between the rotor 400 and the shaft sleeve 520, and the stop member 600 can abut against the shaft sleeve 520 to restrict movement of the rotating shaft 200 towards the impeller 20 along the axis of the rotating shaft 200.

Due to the fact that the stop member 600, the rotating shaft 200, and the rotor 400 rotate and move synchronously, the stop member 600 can abut against the shaft sleeve 520 to restrict the movement of the rotating shaft 200 towards the impeller 20 along the axis of the rotating shaft 200, a side of the sphere 900 facing the rotor 400 abuts against the first spherical wall 4124a of the first groove 4124, and a side of the sphere 900 facing the support member 510 abuts against the second spherical wall 514 of the second groove 512, so that a range of movement of the rotating shaft 200 in a direction away from the impeller 20 along the axis of the rotating shaft 200 is limited, thereby limiting the rotating shaft 200 on the axis of the rotating shaft 200. Meanwhile, the rotating shaft 200 extends through the shaft sleeve 520, and the sphere 900 is arranged between the first groove 4124 and the second groove 512 at the same time, so that the rotating shaft 200 can drive the sphere 900 to roll in the first groove 4124 and the second groove 512 when swinging in the radial direction, and a rolling range of the sphere 900 in the radial direction of the rotating shaft 200 can be limited, thereby overally limiting a radial swinging range of the rotating shaft 200. In other words, the above design realizes both axial limiting and radial limiting of the rotating shaft 200.

Moreover, since the sphere 900 is arranged and a gravity center of the sphere 900 is a sphere center, during assembling, it is only required to cause the rotating axis of the rotating assembly to coincide with the central axis of the cavity defined by the first spherical wall 4124a. First, the rotating shaft 200 is kept in a vertical state, the opening of the first groove 4124 faces upwards, and the sphere 900 is freely placed in the first groove 4124 by means of gravity, such that the sphere 900 and the rotating shaft 200 can be coaxial. Then, the second groove 514 of the support member 510 cooperates with the sphere 900 to finish assembling. The second groove 512 is only required to support and limit the sphere 900, and the central axis of the cavity defined by the second spherical wall 514 of the support member 510 is not required to coincide with the central axis of the sphere 900, so that the assembling difficulty is reduced, and an assembling process is simple and quick.

In the illustrated embodiment, the stop member 600 is fixedly connected to the second rotor unit 420. Specifically, the stop member 600 is fixedly connected to the second flywheel 422 of the second rotor unit 420. In some embodiments, the stop member 600 is bonded to the second flywheel 422 of the second rotor unit 420. In some embodiments, the stop member 600 and the second flywheel 422 of the second rotor unit 420 are integrally formed. Since the blood pump 1 has a small overall size, the stop member 600 has a smaller size, resulting in high machining precision and large assembling difficulty. When the stop member 600 and the second flywheel 422 are integrally formed, the mounting is convenient, and the bonding operation is omitted.

Specifically, when the stop member 600 abuts against the shaft sleeve 520, a gap for fluid circulation is formed between the stop member 600 and an inner wall of the limiting cavity 112, and the shaft sleeve 520 is spaced apart from the rotor 400 by a distance. Since the gap for fluid circulation is formed between the stop member 600 and the inner wall of the limiting cavity 112, the flushing liquid can flow into the gap between the hole wall of the shaft hole 522 of the shaft sleeve 520 and the rotating shaft 200 via the gap between the stop member 600 and the inner wall of the limiting cavity 112. That is, fluid communication between the shaft hole 522 of the shaft sleeve 520 and the receiving cavity 114 is realized. When the stop member 600 abuts against the shaft sleeve 520, the shaft sleeve 520 is spaced apart from the rotor 400 by a distance to avoid that the rotor 400 directly contacts the shaft sleeve 520 to rub against the shaft sleeve 520 to cause abrasion, that is, to avoid abrasion between the second rotor unit 420 and the shaft sleeve 520.

Specifically, the stop member 600 is substantially annular. A central axis of the stop member 600 coincides with the axis of the rotating shaft 200. The stop member 600 has an outer diameter less than an inner diameter of the limiting cavity 112, so that the gap for fluid circulation is formed between the stop member 600 and the inner wall of the limiting cavity 112. In other embodiments, the stop member 600 may alternatively be formed by arranging a plurality of fan-shaped rings which are arranged in a circle around the rotating shaft 200 at an even interval, or the stop member 600 may be understood to be formed by arranging a plurality of fan-shaped rings which are discretely arranged in a circumferential direction.

Specifically, the shaft sleeve 520 is provided with a third groove 523. The third groove 523 has a concave third spherical wall 523a. The stop member 600 has a convex stop surface 610. The stop member 600 is partially arranged in the third groove 523, so that the stop surface 610 abuts against the third spherical wall 523a. A shape of the convex stop surface 610 matches with a shape of the concave third spherical wall 523a, and the third spherical wall 523a can abut against the stop surface 610 to limit the movement of the rotating shaft 200 along the axis of the rotating shaft 200 towards the impeller 20. Contact surfaces of the stop surface 610 and the third spherical wall 523a are arc-shaped, and an arc-arc contact is enabled, with a large contact area, and the little abrasion. More specifically, a diameter of a cavity defined by the third spherical wall 523a is less than a diameter of the shaft sleeve 520, so that the third groove 523 has a certain radial limiting effect on the stop member 600.

Specifically, a thickness of the stop member 600 along the axis of the rotating shaft 200 is greater than a length of the third groove 523 along the axis of the rotating shaft 200, so that the shaft sleeve 520 is spaced apart from the rotor 400 (specifically, the second rotor unit 420) by a distance when the stop member 600 abuts against the shaft sleeve 520. It may be understood that, in some embodiments, the thickness of the stop member 600 along the axis of the rotating shaft 200 may alternatively be less than or equal to the length of the third groove 523 along the axis of the rotating shaft 200, and in this case, the rotor 400 (specifically, the second rotor unit 420) and the stop member 600 may be spaced apart by a distance along the axis of the rotating shaft 200, and the distance is sufficient to allow the shaft sleeve 520 to be spaced apart from the rotor 400 by a distance when the stop member 600 abuts against the shaft sleeve 520.

Specifically, a surface of the shaft sleeve 520 facing the stop member 600 is partially recessed to form a flow guide groove 524. The flow guide groove 524 is in communication with the shaft hole 522 of the shaft sleeve 520. When the stop member 600 abuts against the shaft sleeve 520, a part of the flow guide groove 524 is not covered by the stop member 600, so that when the stop member 600 abuts against the shaft sleeve 520, even if the stop member 600 blocks the gap between the shaft hole 522 of the shaft sleeve 520 and the rotating shaft 200 to cause a problem of flow obstruction of the flushing liquid, the flow guide groove 524 which is not covered by the stop member 600 can realize fluid communication when the stop member 600 abuts against the shaft sleeve 520, such that smoothness of circulation of the flushing liquid is ensured. In addition, the flow guide groove 524 is formed by partially recessing the surface of the shaft sleeve 520 facing the stop member 600, so that the flushing liquid can better flow into a space between the stop member 600 and the shaft sleeve 520, thereby playing a role of lubricating contact surfaces of the stop member 600 and the shaft sleeve 520, reducing the friction between the stop member 600 and the shaft sleeve 520, and reducing the abrasion caused by the friction between the stop member 600 and the shaft sleeve 520.

Specifically, roughness of at least one of the stop surface 610 and the third spherical wall 523a is less than or equal to 0.1 microns. In some embodiments, the roughness of the stop surface 610 and the roughness of the third spherical wall 523a are less than or equal to 0.1 microns. In some embodiments, the roughness of one of the stop surface 610 and the third spherical wall 523a is less than or equal to 0.1 microns. By reducing the roughness of at least one of the stop surface 610 and the third spherical wall 523a, a friction force between the stop surface 610 and the third spherical wall 523a can be effectively reduced, and the abrasion caused by the friction between the shaft sleeve 520 and the stop member 600 can be reduced.

In some embodiments, at least one of the stop surface 610 and the third spherical wall 523a is a ceramic surface. Ceramic has high processing precision, high biocompatibility, high mechanical strength, better abrasion resistance, and better corrosion resistance. In this case, the stop member 600 and the shaft sleeve 520 may be made of ceramic, and alternatively, at least one of the stop surface 610 and the third spherical wall 523a may be a ceramic surface by providing a ceramic coating. In some embodiments, the stop surface 610 is made of diamond, so that the stop surface 610 has high hardness and is relatively smooth and resistant to abrasion, and in this case, the stop surface 610 is a ceramic surface in material by providing a diamond coating

In some embodiments, at least one of the rotating shaft 200, the shaft sleeve 520, the support member 510, and the sphere 900 is made of ceramic material. Compared with a metal material, the ceramic has high processing precision, biocompatibility and mechanical strength, and has better abrasion resistance and corrosion resistance. Alternatively, roughness of at least one of the rotating shaft 200, the shaft sleeve 520, the support member 510, and the sphere 900 is less than or equal to 0.1 microns.

It may be understood that the structure of the driving mechanism 10 is not limited to the above-described structure. In some embodiments, one rotor unit of the rotor 400 and one stator unit of the stator 300 are provided, and in this case, the rotor unit is arranged proximate to the shaft sleeve 520, and the stator unit is arranged proximate to the support member 510. In some embodiments, the rotor 400 still has the first rotor unit 410 and the second rotor unit 420, but the stator 300 has one stator unit, and in this case, the stator unit is located between the first rotor unit 410 and the second rotor unit 420, and the stator unit can simultaneously drive the first rotor unit 410 and the second rotor unit 420 to rotate.

The above embodiments are only used to illustrate the technical solutions of the present disclosure, not to limit the present disclosure; although the present disclosure is described in detail with reference to the above embodiments, those having ordinary skill in the art should understand that they still can modify technical solutions recited in the aforesaid embodiments or equivalently replace part of technical features therein; these modifications or replacements do not make essence of corresponding technical solutions depart from the spirit and scope of technical solutions of embodiments of the present disclosure, and all of them should be included in the protection scope of the present disclosure.

1. A driving mechanism, comprising:
a housing assembly;
a rotating assembly having a distal end and a proximal end, the distal end of the rotating assembly being rotatably mounted on the housing assembly, the proximal end of the rotating assembly being provided with a first groove, and the first groove having a concave first spherical wall, wherein the housing assembly is provided with a second groove, the second groove being opposed to the first groove, and the second groove having a concave second spherical wall; and
a sphere partially arranged in the first groove and partially arranged in the second groove, and the sphere respectively slidably abutting against the first spherical wall and the second spherical wall.

2. The driving mechanism according to claim 1, wherein each of an opening edge of the first groove proximate to the second groove and an opening edge of the second groove proximate to the first groove is provided with a rounded portion.

3. The driving mechanism according to claim 1, wherein a diameter of the sphere is greater than a sum of lengths of the first groove and the second groove along a rotating axis of the rotating assembly.

4. The driving mechanism according to claim 3, wherein the length of the first groove along the rotating axis of the rotating assembly is greater than or equal to ¼ of the diameter of the sphere and less than ½ of the diameter of the sphere;
and/or, the length of the second groove along the rotating axis of the rotating assembly is greater than or equal to ¼ of the diameter of the sphere and less than ½ of the diameter of the sphere.

5. The driving mechanism according to claim 1, wherein the second groove has a first opening and a second opening, the first opening being closer to the first groove than the second opening, and a central axis of the first opening coinciding with a central axis of the second opening;
wherein the second opening is centrally located at the second spherical wall; and/or a diameter of the second opening is 1/9 to ⅓ of the diameter of the sphere.

6. The driving mechanism according to claim 1, wherein the housing assembly comprises a pump case and a support member mounted on the pump case; the second groove is formed in the support member; wherein the driving mechanism further comprises a support base fixedly connected to the pump case, wherein a mounting cavity and a liquid passing hole in communication with the mounting cavity are formed on the support base, the support member is mounted in the mounting cavity, and the second groove is in communication with the liquid passing hole.

7. The driving mechanism according to claim 6, wherein the mounting cavity has a cavity bottom, an opening of the liquid passing hole is located at the cavity bottom, a support step is arranged in the mounting cavity; and the support step abuts against the support member, so that the support member is spaced apart from the cavity bottom by a distance;
and/or, the support base is further provided with a flow diversion channel, and the flow diversion channel being in communication with the liquid passing hole such that fluid entering the liquid passing hole is also capable of flowing into the pump case via the flow diversion channel;
and/or, the second groove has a first opening and a second opening, the first opening being opposite to the first groove, the second opening being in communication with the liquid passing hole; the support member is further provided with a communication hole, wherein the communication hole is in communication with the second opening and the liquid passing hole, and the communication hole has a certain length along a central axis of the first opening.

8. The driving mechanism according to claim 6, wherein the support member is further provided with a communication hole in communication with the second groove, and the communication hole being in communication with the liquid passing hole;
wherein the communication hole is a straight hole; and/or a central axis of the communication hole coincides with a central axis of a cavity defined by the second spherical wall.

9. The driving mechanism according to claim 6, wherein an opening of a side of the first groove proximate to the second groove and an opening of a side of the second groove proximate to the first groove are spaced apart by a distance, and the distance enables the rotating assembly and the support member to be kept spaced apart, to avoid that the rotating assembly rubs against the support member when the rotating assembly swings radially.

10. The driving mechanism according to claim 1, wherein the housing assembly comprises a pump case and a shaft sleeve mounted on the pump case; a distal end of the rotating assembly rotatably extends through the shaft sleeve; wherein the driving mechanism further comprises a stop member fixedly connected to the rotating assembly, wherein the stop member is located between the shaft sleeve and the sphere, and the stop member is capable of abutting against the shaft sleeve to prevent the rotating assembly from moving away from the sphere.

11. The driving mechanism according to claim 10, wherein the shaft sleeve is provided with a shaft hole, the rotating assembly rotatably extends through the shaft hole; a surface of the shaft sleeve facing the stop member is locally recessed to form a flow guide groove, and the flow guide groove being in communication with the shaft hole; when the stop member abuts against the shaft sleeve, a part of the flow guide groove is not covered by the stop member.

12. The driving mechanism according to claim 11, wherein the pump case has an inner cavity, and wherein the inner cavity comprises a limiting cavity and a receiving cavity which are arranged in an axial direction of the pump case; wherein when the stop member abuts against the shaft sleeve, a gap for fluid circulation is formed between the stop member and an inner wall of the limiting cavity.

13. The driving mechanism according to claim 10, wherein the shaft sleeve is provided with a third groove having a concave third spherical wall; the stop member has a convex stop surface capable of abutting against the third spherical wall.

14. The driving mechanism according to claim 13, further comprising a rotating shaft rotatably mounted on the pump case, wherein a thickness of the stop member along an axis of the rotating shaft is greater than a length of the third groove along the axis of the rotating shaft.

15. The driving mechanism according to claim 1, wherein the housing assembly comprises a pump case, a support member, and a shaft sleeve, wherein the support member and the shaft sleeve are mounted on the pump case, the second groove is formed in the support member; a distal end of the rotating assembly rotatably extends through the shaft sleeve; the sphere is movably received in the pump case; the support member, the shaft sleeve, and the sphere are arranged along a rotating axis of the rotating assembly; and the support member, the shaft sleeve, and the sphere are capable of collectively limit the rotating assembly.

16. The driving mechanism according to claim 15, wherein the rotating assembly comprises: a rotating shaft rotatably extending through the shaft sleeve; and a rotor fixedly connected to the rotating shaft; wherein the driving mechanism further comprises a stator, wherein the stator and the rotor are located between the support member and the shaft sleeve, and the stator is capable of generating a rotating magnetic field to drive the rotor to rotate.

17. The driving mechanism according to claim 1, wherein the rotating assembly comprises: a rotating shaft having a proximal end and a distal end; and a rotor comprising a first rotor unit, wherein the distal end of the rotating shaft is rotatably mounted on the housing assembly, the first rotor unit is fixedly connected to the proximal end of the rotating shaft, and the first groove is formed in the first rotor unit.

18. The driving mechanism according to claim 17, wherein the rotor further comprises a second rotor unit fixedly connected to the rotating shaft and arranged proximate to the distal end of the rotating shaft; wherein the driving mechanism further comprises a stator, the stator comprising a first stator unit and a second stator unit which are arranged along an axis of the rotating shaft, wherein the first stator unit and the second stator unit are located between the first rotor unit and the second rotor unit; the first stator unit is capable of driving the first rotor unit to rotate, and the second stator unit is capable of driving the second rotor unit to rotate; each of the first stator unit and the second stator unit comprises a magnetic core and a coil wound around the magnetic core;
the driving mechanism further comprises a magnetic conductive member connected to the housing assembly; the magnetic core of the first stator unit and the magnetic core of the second stator unit are fixedly connected to the magnetic conductive member; and the rotating shaft rotatably extends through the first stator unit, the second stator unit, and the magnetic conductive member.

19. A blood pump, comprising an impeller and a driving mechanism, the driving mechanism comprising:
a housing assembly;
a rotating assembly having a distal end and a proximal end, the distal end of the rotating assembly being rotatably mounted on the housing assembly, the proximal end of the rotating assembly being provided with a first groove, and the first groove having a concave first spherical wall, wherein the housing assembly is provided with a second groove, the second groove being opposed to the first groove, and the second groove having a concave second spherical wall; and
a sphere partially arranged in the first groove and partially arranged in the second groove, and wherein the sphere respectively slidably abuts against the first spherical wall and the second spherical wall; the impeller is connected to the rotating assembly, and rotatable along with the rotating assembly.
